(19) United States
(12) Patent Application Publication
(10) Pub. No.: US 2025/0312620 A1
(43) Pub. Date: Oct. 9, 2025

(54) NEUTRON CAPTURE THERAPY APPARATUS

(71) Applicant: SUMITOMO HEAVY INDUSTRIES, LTD., Tokyo (JP)

(72) Inventor: Kenzo SASAI, Niihama-shi (JP)

(21) Appl. No.: 19/243,680

(22) Filed: Jun. 19, 2025

Related U.S. Application Data
(63) Continuation of application No. PCT/JP2023/046471, filed on Dec. 25, 2023.

(30) Foreign Application Priority Data
Dec. 28, 2022 (JP) 2022-211238

(51) Int. Cl. A61N 5/10

(52) U.S. Cl. CPC A61N 5/103; A61N 2005/109

(57) ABSTRACT

A neutron capture therapy apparatus includes: an irradiator that irradiates an irradiation spot of an irradiation target with a neutron ray; and an electromagnetic wave irradiator that irradiates the irradiation target with an electromagnetic wave for aligning the irradiation target with a position where the irradiation target is disposed, in which the electromagnetic wave irradiator changes an irradiation position of the electromagnetic wave based on treatment plan information regarding the irradiation target.

### CROSS-REFERENCE TO RELATED APPLICATIONS

This is a bypass continuation of International PCT Application No. PCT/JP2023/046471, filed on Dec. 25, 2023, which claims priority to Japanese Patent Application No. 2022-211238, filed on Dec. 28, 2022, which are incorporated by reference herein in their entirety.

### BACKGROUND

### Technical Field

A certain embodiment of the present disclosure relates to a neutron capture therapy apparatus.

### Description of Related Art

As a neutron capture therapy apparatus that emits a neutron ray to kill cancer cells, a boron neutron capture therapy (BNCT) using a boron compound as shown in the related art is known. In the boron neutron capture therapy, boron that has been previously incorporated into the cancer cells is irradiated with the neutron ray to selectively destroy the cancer cells using scattering of heavy charged particles generated by the irradiation.

### SUMMARY

According to an embodiment, there is provided a neutron capture therapy apparatus including: an irradiator that irradiates an irradiation spot of an irradiation target with a neutron ray; a treatment plan acquisition unit that acquires treatment plan information regarding the irradiation target; and an electromagnetic wave irradiator that irradiates the irradiation target with an electromagnetic wave for aligning the irradiation target with a position where the irradiation target is disposed. The electromagnetic wave irradiator changes an irradiation position of the electromagnetic wave based on treatment plan information acquired by the treatment plan acquisition unit.

### BRIEF DESCRIPTION OF THE DRAWINGS

FIG. 1 is a schematic diagram illustrating a neutron capture therapy apparatus according to an embodiment of the present disclosure.

FIG. 2 is a conceptual diagram illustrating an electromagnetic wave irradiator and a block configuration of the neutron capture therapy apparatus.

FIGS. 3A and 3B are views illustrating the electromagnetic wave irradiator as seen from each direction.

FIG. 4 is a conceptual diagram illustrating an example of a positional relationship between the affected part and a collimator.

### DETAILED DESCRIPTION

Here, in the neutron capture therapy apparatus, the affected part of the patient (irradiation spot of the irradiation target) is aligned with an irradiator that emits the neutron ray in accordance with a treatment planning prepared in advance. Accordingly, the treatment is possible in accordance with the treatment planning. In the neutron capture therapy apparatus, a situation during treatment may be different for each treatment, such as a length of a collimator of an irradiator, a distance at which a patient can be close to the irradiator, a position of an affected part in the patient, and a posture of the patient. In this manner, there is a problem in that the positional relationship between the reference position of the irradiator and the affected part deviates from the treatment plan depending on the situation during the treatment.

Therefore, it is desirable to provide a neutron capture therapy apparatus that can perform treatment according to a treatment planning regardless of a situation during treatment.

The neutron capture therapy apparatus according to the embodiment includes an electromagnetic wave irradiator that emits electromagnetic waves for aligning the irradiation target with a position where the irradiation target is disposed. Therefore, before the treatment, the irradiation target can be accurately disposed at the position designated in the treatment planning by aligning the irradiation target based on the irradiation position irradiated with the electromagnetic wave. In addition, the electromagnetic wave irradiator changes the irradiation position of the electromagnetic wave, based on the treatment plan information acquired by the treatment plan acquisition unit. In this manner, the irradiation position of the electromagnetic wave can be changed. Therefore, for example, the irradiation position can be adjusted to an appropriate position according to a situation during the treatment, such as a length of a collimator or a position of an irradiation spot on an irradiation target. Further, the electromagnetic wave irradiator can adjust the irradiation position based on the treatment plan information. Therefore, the irradiation position of the electromagnetic wave can be appropriately adjusted after the situation during the treatment is identified from the treatment plan information. From the above, the treatment according to the treatment planning can be performed regardless of the situation during the treatment.

The electromagnetic wave irradiator may be a laser irradiation unit. In this case, the alignment of the irradiation target can be easily performed while visually confirming the position irradiated with the laser.

The treatment plan information may include at least information for calculating a distance and a direction between a reference position on a side of the irradiator that emits the neutron ray and the irradiation spot. Accordingly, the electromagnetic wave irradiator is capable of adjusting the irradiation position of the electromagnetic wave to be able to reproduce the distance and the direction between the reference position and the irradiation spot.

The electromagnetic wave irradiator may change the irradiation position of the electromagnetic wave along an axial direction in which a center axis of the neutron ray extends. In this case, the irradiation target can be accurately aligned in the axial direction in which the center axis extends.

The electromagnetic wave irradiator may change the irradiation position along a direction perpendicular to an axial direction in which a center axis of the neutron ray extends. In this case, the irradiation target can be accurately aligned in the direction perpendicular to the axial direction in which the center axis extends.

The irradiation position of the electromagnetic wave irradiator may be recalculated based on information regarding an irradiation field forming device that forms an irradiation field of the neutron ray. In this case, the irradiation position of the electromagnetic wave irradiator can be adjusted to an appropriate position in consideration of the structure of the irradiation field forming device.

Hereinafter, a preferred embodiment of the present disclosure will be described in detail with reference to drawings.

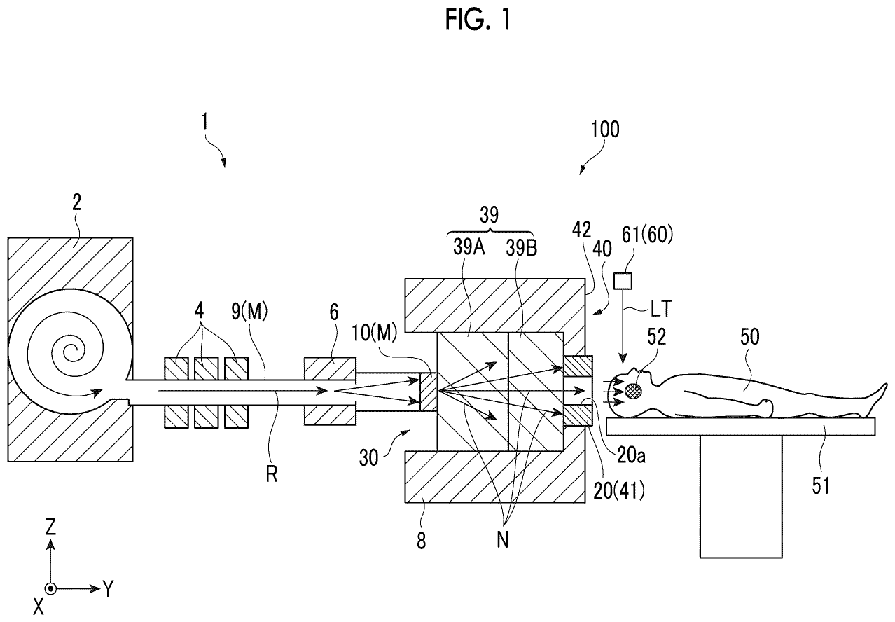

First, an outline of a neutron capture therapy apparatus according to an embodiment of the present disclosure will be described with reference to FIG. 1. A neutron capture therapy apparatus 1 illustrated in FIG. 1 is an apparatus that performs cancer treatment using boron neutron capture therapy (BNCT). In the neutron capture therapy apparatus 1, for example, the irradiation with the neutron ray N is performed on an affected part 52 (irradiation spot) of a patient 50 (irradiation target) on a treatment table 51 to which boron (10B) is administered. The affected part 52 is a tumor formed in the patient 50.

The neutron capture therapy apparatus 1 includes an accelerator 2. The accelerator 2 accelerates charged particles such as negative ions to emit a charged particle beam R. The accelerator 2 is formed of, for example, a cyclotron. In the present embodiment, the charged particle beam R is a proton beam that is generated by stripping electrons from negative ions. The accelerator 2 generates the charged particle beam R having, for example, a beam radius of 40 mm and a power of 60 KW (=30 MeV×2 mA). The accelerator is not limited to the cyclotron, and may be a synchrotron, a synchrocyclotron, a linear accelerator, an electrostatic accelerator, or the like.

The charged particle beam R emitted from the accelerator 2 is sent to a neutron ray generating unit M. The neutron ray generating unit M includes a beam duct 9 (transport path) and a target 10. The charged particle beam R emitted from the accelerator 2 passes through the beam duct 9 and proceeds toward the target 10 disposed at an end portion of the beam duct 9. A plurality of quadrupole electromagnets 4 and a scanning electromagnet 6 are provided along the beam duct 9. The plurality of quadrupole electromagnets 4 are used, for example, to adjust the beam diameter of the charged particle beam R using an electromagnet.

The scanning electromagnet 6 is to scan the charged particle beam R and to control the irradiation of the target 10 with the charged particle beam R. The scanning electromagnet 6 controls the irradiation position of the target 10 with the charged particle beam R.

The neutron capture therapy apparatus 1 includes a neutron generating unit 100 that irradiates the target 10 with the charged particle beam R to generate the neutron rays N and emits the neutron rays N toward the patient 50. The neutron generating unit 100 includes the target 10, a shield member 8, a deceleration member 39, and an irradiator 40.

The target 10 is to generate the neutron rays N in a case where the target 10 is irradiated with the charged particle beam R. The target 10 is a solid member made of a material that generates neutron rays by being irradiated with the charged particle beam. Specifically, the target 10 is made of, for example, beryllium (Be), lithium (Li), tantalum (Ta), or tungsten (W), and has, for example, a disk-shaped solid shape having a diameter of 160 mm. The target 10 is not limited to the disk shape, and may have another shape.

The deceleration member 39 decelerates the neutron ray N generated by the target 10 (decreases energy of the neutron ray N). The deceleration member 39 may have a laminated structure consisting of a layer 39A that mainly decelerates fast neutrons contained in the neutron ray N, and a layer 39B that mainly decelerates epithermal neutrons contained in the neutron ray N. A substance having a large total cross-sectional area for neutrons having an energy of 40 keV or more is employed as the material of the deceleration member 39. Specifically, examples of the material of the deceleration member 39 include CaF₂, MgF₂, AlF₃, D₂O, and polyethylene.

The shield member 8 shields the generated neutron ray N, a gamma ray generated due to the generation of the neutron ray N, and the like not to be released to the outside. The shield member 8 is provided to surround the deceleration member 39. An upper portion and a lower portion of the shield member 8 extend to an upstream side of the charged particle beam R more than the deceleration member 39. As the material for the neutron shielding material of the shield member 8, a substance having a large total cross-sectional area for neutrons of 0.5 eV or less is employed. Examples of the material of the neutron shielding material include Li, boron, cadmium, and polyethylene. As the material for the gamma ray shielding material of the shield member 8, a substance having a large total cross-sectional area for gamma rays is employed. Examples of the material of the gamma ray shielding material include lead and iron.

The irradiator 40 irradiates the affected part 52 with the neutron ray N of the patient 50. The irradiator 40 is disposed on the downstream side of the deceleration member 39 with respect to the neutron ray N, and irradiates the patient 50 with the neutron ray N that has passed through the deceleration member 39. The irradiator 40 includes an irradiation field forming device 41 formed on a wall portion 42 of the treatment room. The irradiation field forming device 41 is a device for forming an irradiation field of the neutron ray N according to a shape of the affected part 52 of the patient 50 or the like. In the present embodiment, the irradiation field forming device 41 includes the collimator 20. The collimator 20 is provided in the wall portion 42 and includes a hole portion 20a for emitting the neutron ray N. The collimator 20 forms an irradiation field of the neutron ray N by emitting the neutron ray N passing through the hole portion 20a and restricting the emission of the neutron ray N other than the neutron ray N passing through the hole portion 20a.

The neutron capture therapy apparatus 1 includes an electromagnetic wave irradiator 60. The electromagnetic wave irradiator 60 irradiates the position where the patient 50 is disposed with electromagnetic waves for alignment of the patient 50. The electromagnetic waves are not particularly limited as long as the electromagnetic waves enable alignment of the patient 50 by being irradiated onto the patient 50. Visible light that is emitted in a state of having directivity as electromagnetic waves may be adopted. In the present embodiment, a laser irradiation unit 61 that irradiates the laser LT is adopted as the electromagnetic wave irradiator 60. The electromagnetic waves are not limited to visible light such as the laser LT, and radio waves or the like may also be employed. The electromagnetic wave irradiator 60 may irradiate the patient 50 with a radio wave having directivity and may identify a site of the patient 50 by a reflected wave.

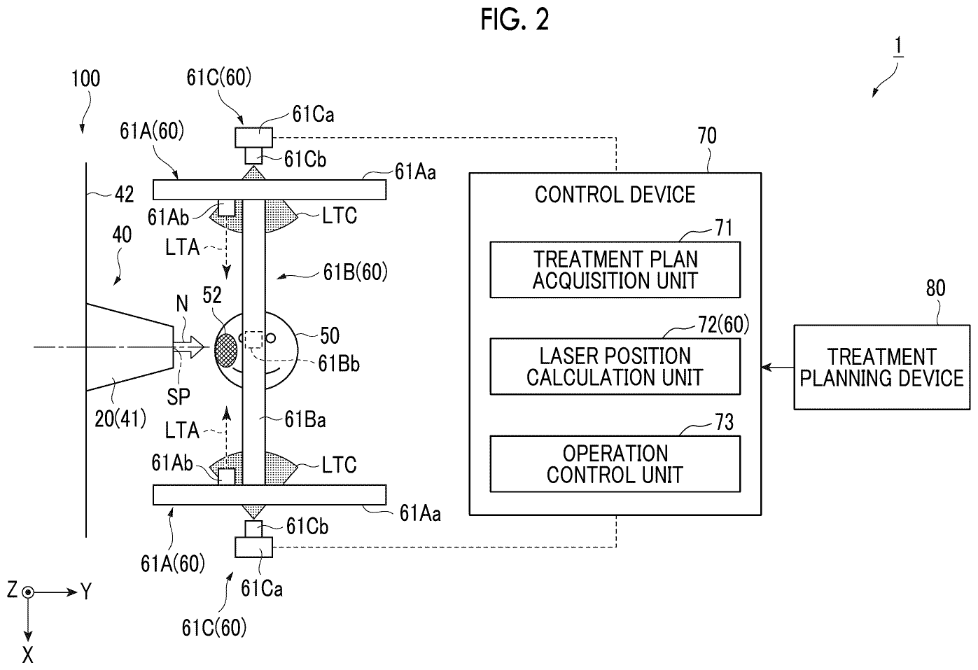

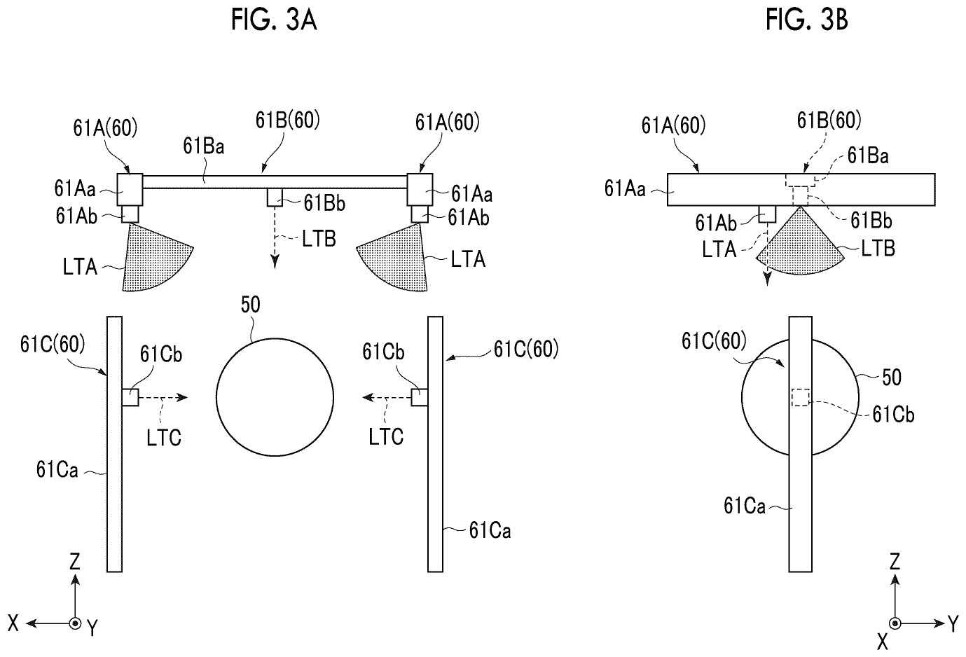

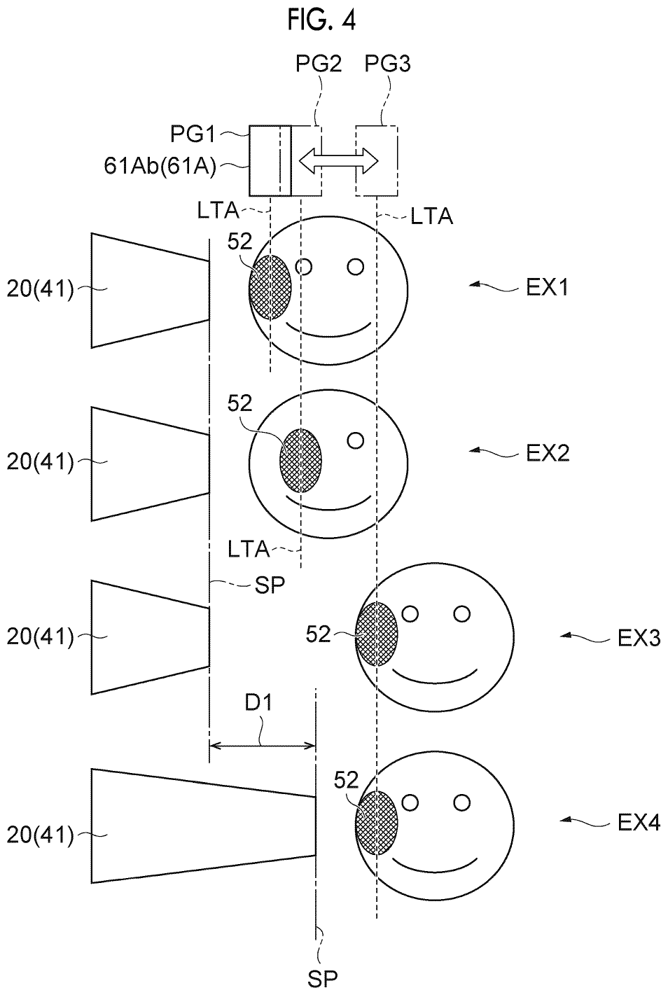

Next, the neutron capture therapy apparatus 1 will be described in more detail with reference to FIGS. 2 to 4. FIG. 2 is a conceptual diagram illustrating the electromagnetic wave irradiator 60 and the block configuration of the neutron capture therapy apparatus 1. FIGS. 3A and 3B are views of the electromagnetic wave irradiator 60 as seen from each direction. FIG. 4 is a conceptual diagram illustrating an example of a positional relationship between the affected part 52 and the collimator 20. In FIG. 4, a plurality of positional relationships of Mode 1 to Mode 4 are described side by side. In addition, in the following description, the center axis of the neutron ray N may be referred to as CL. The center axis CL is a center position in the irradiation with the neutron ray N. A direction parallel to the center axis CL is defined as a Y-axis direction. The Y-axis direction is an axial direction in which the center axis CL extends. A direction in which the neutron ray N proceeds is defined as a positive side in a Y-axis direction. A horizontal direction perpendicular to the center axis CL is defined as an X-axis direction. One side in the X-axis direction is defined as a positive side. The up-down direction is defined as a Z-axis direction. The upper side is a positive side in the Z-axis direction. The collimator 20 is provided to protrude from the wall portion 42 to the positive side in the Y-axis direction. The position of the center axis CL is set to the reference position SP of the irradiator 40 at the end portion on the positive side in the Y-axis direction of the collimator 20. The reference position SP may be set at any position on the irradiator 40 side as long as the reference position SP is a position that is a reference for expressing a relative positional relationship between the affected part 52 of the patient 50 and the irradiator 40.

As illustrated in FIGS. 2, 3A, and 3B, in the neutron capture therapy apparatus 1, the laser irradiation unit 61 includes a pair of Y-axis laser irradiation units 61A moving in the Y-axis direction, an X-axis laser irradiation unit 61B moving in the X-axis direction, and a pair of Z-axis laser irradiation units 61C moving in the Z-axis direction.

The pair of Y-axis laser irradiation units 61A includes a guide portion 61Aa and a laser irradiator 61Ab. The guide portion 61Aa extends in the Y-axis direction. The pair of guide portions 61Aa are disposed at positions separated from each other in the X-axis direction to sandwich the patient 50. The laser irradiator 61Ab irradiates the laser LTA. The laser irradiator 61Ab irradiates the center axis CL side with the laser LTA in the X-axis direction. The laser irradiator 61Ab emits the laser LTA to be parallel to the X-axis direction when viewed from the Z-axis direction (refer to FIG. 2). The laser irradiator 61Ab emits the laser LTA to spread in a fan shape when viewed from the Y-axis direction (refer to FIG. 3A). In this manner, the pair of Y-axis laser irradiation units 61A can indicate the target position in the Y-axis direction of the affected part 52 by the laser LTA. In addition, the laser irradiator 61Ab of the Y-axis laser irradiation unit 61A is movable in the Y-axis direction along the guide portion 61Aa. The laser irradiator 61Ab is movable in the Y-axis direction in a state where the irradiating direction of the laser LTA is fixed. In this manner, the Y-axis laser irradiation unit 61A can change the position of the origin of the laser LTA (that is, the laser irradiator 61Ab) in the Y-axis direction. Therefore, the laser LTA can change the target position in the Y-axis direction of the affected part 52, in the Y-axis direction.

The X-axis laser irradiation unit 61B includes a guide portion 61Ba and a laser irradiator 61Bb. The guide portion 61Ba extends in the X-axis direction. The guide portion 61Ba is provided to be bridged between the guide portions 61Aa of the pair of Y-axis laser irradiation units 61A. The laser irradiator 61Bb irradiates the laser LTB. The laser irradiator 61Bb irradiates the negative side in the Z-axis direction with the laser LTB. The laser irradiator 61Bb emits the laser LTB to be parallel to the Z-axis direction when viewed from the Y-axis direction (refer to FIG. 3A). The laser irradiator 61Bb emits the laser LTB to spread in a fan shape when viewed from the X-axis direction (refer to FIG. 3B). In this manner, the X-axis laser irradiation unit 61B can indicate the target position of the affected part 52 in the X-axis direction by the laser LTB. In addition, the laser irradiator 61Bb of the X-axis laser irradiation unit 61B is movable in the X-axis direction along the guide portion 61Ba. The laser irradiator 61Bb is movable in the X-axis direction in a state where the irradiating direction of the laser LTB is fixed. In this manner, the X-axis laser irradiation unit 61B can change the position of the origin of the laser LTB (that is, the laser irradiator 61Bb) in the X-axis direction. Therefore, the laser LTB can change the target position in the X-axis direction of the affected part 52, in the X-axis direction.

The pair of Z-axis laser irradiation units 61C includes a guide portion 61Ca and a laser irradiator 61Cb. The guide portion 61Ca extends in the Z-axis direction. The pair of guide portions 61Ca are disposed at positions separated from each other in the X-axis direction to sandwich the patient 50. The laser irradiator 61Cb irradiates the laser LTC. The laser irradiator 61Cb irradiates the center axis CL side with the laser LTC in the X-axis direction. The laser irradiator 61Cb emits the laser LTC to be parallel to the X-axis direction when viewed in the Y-axis direction (refer to FIG. 3A). The laser irradiator 61Cb emits the laser LTC to spread in a fan shape when viewed in the Z-axis direction (refer to FIG. 2). In this manner, the Z-axis laser irradiation unit 61C can indicate the target position of the affected part 52 in the Z-axis direction by the laser LTC. In addition, the laser irradiator 61Cb of the Z-axis laser irradiation unit 61C is movable in the Z-axis direction along the guide portion 61Ca. The laser irradiator 61Cb is movable in the Z-axis direction in a state where the irradiating direction of the laser LTC is fixed. In this manner, the Z-axis laser irradiation unit 61C can change the position of the origin of the laser LTC (that is, the laser irradiator 61Cb) in the Z-axis direction. Therefore, the laser LTC can change the target position in the Z-axis direction of the affected part 52, in the Z-axis direction.

As shown in FIG. 2, the neutron capture therapy apparatus 1 includes a control device 70. The control device 70 is a device that controls the entirety of the neutron capture therapy apparatus 1. The control device 70 includes a treatment plan acquisition unit 71, a laser position calculation unit 72, and an operation control unit 73.

The treatment plan acquisition unit 71 acquires the treatment planning from a treatment planning device 80. The treatment planning device 80 is a device indicating how to perform the treatment with the neutron capture therapy apparatus 1. The treatment plan information includes at least information on the distance and the direction between the reference position SP on the irradiator 40 side and the affected part 52.

The treatment plan information includes information from which the distance and the direction between the reference position SP and the patient 50 can be calculated. For example, as shown in the Modes EX1, EX2, and EX4 in FIG. 4, there is a case where the face of the patient 50 is at a distance close to the reference position SP of the collimator 20, and as shown in Mode EX3, there is a case where the face of the patient 50 is at a distance at which the face cannot be close to the collimator 20. The treatment plan information includes information on a distance and a direction of the current treatment. The treatment plan information includes information on the position of the affected part 52 in the patient 50. For example, as shown in Modes EX1, EX3, and EX4 in FIG. 4, there is a case where the affected part 52 is positioned on the surface of the face of the patient 50, and as shown in Mode EX2, there is a case where the affected part 52 is positioned deeper within the face of the patient 50. The treatment plan information includes information on at which position the affected part 52 will be disposed on the face of the patient 50 in the current treatment. The treatment plan information includes information on the length of the collimator 20. For example, there are a case where the collimator 20 is short as shown in Modes EX1, EX2, and EX3 in FIG. 4, and a case where the collimator 20 is long as shown in Mode EX4. The treatment plan information includes information on the length of the collimator 20 in the current treatment. The treatment planning device 80 can calculate at least the information on the distance and the direction between the reference position SP on the irradiator 40 side and the affected part 52, based on these pieces of information, and include the information in the treatment plan information. Alternatively, even when the treatment plan information does not directly include information on the distance and the direction between the reference position SP and the affected part 52, it is sufficient as long as such information can be calculated by the control device 70 side based on other information. In addition, the alignment may be performed not only on the affected part 52 but also on the body surface of the patient 50. Accordingly, the treatment plan information may include information for calculating the distance and the direction in the X-axis direction, the distance and the direction in the Y-axis direction, and the distance and the direction in the Z-axis direction between the reference position SP and the body surface of the patient 50.

The laser position calculation unit 72 calculates the irradiation position of the laser LT of the laser irradiation unit 61. The laser position calculation unit 72 configures a part of the electromagnetic wave irradiator 60. The laser position calculation unit 72 can calculate the irradiation position of the laser LT, based on the treatment plan information acquired by the treatment plan acquisition unit 71. The laser position calculation unit 72 can change the irradiation position of the laser LT, based on the treatment plan information acquired by the treatment plan acquisition unit 71. The laser position calculation unit 72 calculates in the X-axis direction, the Y-axis direction, and the Z-axis direction where the affected part 52 should be disposed with respect to the reference position SP, with reference to the treatment plan information. In this manner, the laser position calculation unit 72 calculates the position of the laser irradiator 61Ab of the Y-axis laser irradiation unit 61A, the position of the laser irradiator 61Bb of the X-axis laser irradiation unit 61B, and the position of the laser irradiator 61Cb of the Z-axis laser irradiation unit 61C.

The irradiation position of the electromagnetic wave irradiator 60 is recalculated based on information regarding the irradiation field forming device 41 that forms the irradiation field of the neutron ray N. The irradiation field forming device 41 may include an accessory such as a member for adjusting the depth distribution of the neutron ray N or a member for adjusting the lateral distribution of the neutron ray N. Information such as the type, the position, and the size (thickness) of the accessories is included in the treatment plan information. In a case where these accessories are included, the laser position calculation unit 72 recalculates the irradiation position of the laser LT calculated by considering only the distance and the direction between the reference position SP and the affected part 52 to be able to correct the influence of the accessory.

The operation control unit 73 controls the operation of the neutron capture therapy apparatus 1. The operation control unit 73 controls the laser irradiators 61Ab, 61Bb, and 61Cb to move, based on a calculation result by the laser position calculation unit 72. Then, the patient 50 is aligned such that the affected part 52 is disposed at the position indicated by the laser beams LTA, LTB, and LTC.

Next, the alignment of the patient 50 by the electromagnetic wave irradiator 60 will be described with reference to FIG. 4. Mode EX1 illustrates an example in a case where the face of the patient 50 is disposed at a position close to the collimator 20 and the affected part 52 is present on the surface of the face of the patient 50. Mode EX1 is a typical example. In this case, the laser irradiator 61Ab of the Y-axis laser irradiation unit 61A is disposed at a position PG1, and irradiates the position where the affected part 52 is to be disposed with the laser LTA. Mode EX2 illustrates an example in which the affected part 52 is present deeper within the face of the patient 50 compared to Mode EX1. In this case, the laser irradiator 61Ab is disposed at a position PG2 slightly away from the reference position SP from the position PG1. Mode EX3 illustrates an example in a case where the face of the patient 50 cannot be disposed at a position close to the collimator 20 as in Mode EX1. In this case, the laser irradiator 61Ab is disposed at a position PG3 farther from the reference position SP than the position PG1. Mode EX4 illustrates an example in a case where the collimator 20 longer than the collimator 20 in Mode EX1 is adopted. The reference position SP is disposed at a position shifted by the dimension D1 from Mode EX1. In this case, the laser irradiator 61Ab is disposed at the position PG3 shifted by the dimension D1 from the position PG1.

Next, actions and effects of the neutron capture therapy apparatus 1 according to the present embodiment will be described.

The neutron capture therapy apparatus does not include a rotating gantry, and horizontal or fixed irradiation is common. In addition, unlike the X-rays or the proton beam, the neutron ray N for irradiating the affected part 52 is emitted from the end portion of the collimator 20 and diffuses, such that the intensity thereof is rapidly reduced. Therefore, in order to maintain the dose, the affected part 52 needs to be brought close to the collimator 20 on the order of centimeters. Therefore, in the treatment planning, it is common practice to conventionally define the isocenter (patient positioning reference position) at the end portion of the collimator 20. Therefore, when the length of the collimator 20 is changed, the position of the reference position SP on the treatment planning also moves upstream or downstream in the axial direction (Y-axis direction) of the neutron ray N (for example, refer to Mode EX4 of FIG. 4). Further, the distance at which the affected part 52 and the collimator 20 can be brought close to each other also varies for each patient from the viewpoint of the interference between the patient 50 and the collimator 20, the safety of the patient posture, and the optimization of the dose (for example, refer to Mode EX3 of FIG. 4). In addition, the depth of the affected part 52 also varies depending on the patient (for example, refer to Mode EX2 of FIG. 4). In this manner, in the neutron capture therapy apparatus 1, the position on the center axis CL of the neutron ray N of the affected part 52 is different depending on the patient 50. When the distance and the direction between the collimator 20 and the affected part 52 change, the radiation dose distribution changes. Therefore, it is necessary to correctly determine the position of the patient 50, and it is important where the reference position SP is set. Normally, the collimator 20 having an appropriate length and size is selected according to the affected part 52. In contrast, in the neutron capture therapy apparatus according to the comparative example, there is only one laser irradiation unit for alignment fixed in the Y-axis direction. Therefore, when the collimator 20 having a different length is used, the distance and the direction of the patient 50 from the reference position SP are changed, or the depth of the affected part 52 is changed, the irradiation position of the laser deviates from the affected part. Since the positioning is performed using the deviated laser, the position of the collimator 20 relative to the body surface of the patient 50 is measured by another means. Therefore, the positioning takes time and the accuracy is also degraded.

As another comparative example, a neutron capture therapy apparatus including a laser of which the position in the Y-axis direction can be changed will be described. According to the comparative example, the position of the laser can be changed to be aligned with the target position of the affected part 52. However, in reality, the neutron capture therapy apparatus does not include information on the location of the affected part 52, and thus there is a problem in that it is not known to which position the laser should be shifted.

In contrast, the neutron capture therapy apparatus 1 according to the present embodiment includes an electromagnetic wave irradiator 60 that emits electromagnetic waves for aligning the patient 50 with a position where the patient 50 is disposed. Therefore, before the treatment, the patient 50 can be accurately disposed at the position designated in the treatment planning by aligning the patient 50 based on the irradiation position irradiated with the electromagnetic waves. In addition, the electromagnetic wave irradiator 60 can change the irradiation position of the electromagnetic wave, based on the treatment plan information acquired by the treatment plan acquisition unit 71. In this manner, the irradiation position of the electromagnetic wave can be changed. Therefore, for example, the irradiation position can be adjusted to an appropriate position according to a situation during the treatment such as a length of the collimator 20, a position of an irradiation spot in the patient 50, and a posture of the patient 50. Further, the electromagnetic wave irradiator 60 can adjust the irradiation position based on the treatment plan information. Therefore, the irradiation position of the electromagnetic wave can be appropriately adjusted after the situation during the treatment is identified from the treatment plan information. From the above, the treatment according to the treatment planning can be performed regardless of the situation during the treatment.

The electromagnetic wave irradiator 60 may be the laser irradiation unit 61. In this case, the position of the patient 50 can be easily aligned while visually confirming the position irradiated with the laser LT.

The treatment plan information may include at least information for calculating the distance and the direction between the reference position SP on the irradiator 40 side that emits the neutron ray N and the affected part 52. In this manner, the electromagnetic wave irradiator 60 can adjust the irradiation position of the electromagnetic wave to be able to reproduce the distance and the direction between the reference position SP and the affected part 52.

The electromagnetic wave irradiator 60 may be capable of changing the irradiation position of the electromagnetic wave along the axial direction in which the center axis CL of the neutron ray N extends. In this case, the alignment of the affected part 52 in the axial direction in which the center axis CL extends can be accurately performed.

The electromagnetic wave irradiator 60 may be capable of changing the irradiation position along a direction perpendicular to the axial direction along which the center axis CL of the neutron ray N extends. In this case, the patient 50 can be accurately aligned in the direction perpendicular to the axial direction in which the center axis CL extends.

The irradiation position of the electromagnetic wave irradiator 60 may be recalculated based on information regarding the irradiation field forming device 41 that forms the irradiation field of the neutron ray N. In this case, the irradiation position of the electromagnetic wave irradiator 60 can be adjusted to an appropriate position in consideration of the structure of the irradiation field forming device 41.

In the above-described embodiment, the information regarding the irradiation field forming device 41 has been described as being included in the treatment plan information. However, a configuration may be adopted in which the calculation is performed by additionally considering equipment information that is not included in the treatment plan information. For example, the calculation may be further performed using the accessory information that is not included in the treatment plan information.

The present disclosure is not limited to the embodiment described above.

For example, the configuration of the neutron capture therapy apparatus 1 described above is merely an example and can be appropriately changed.

It should be understood that the invention is not limited to the above-described embodiment, but may be modified into various forms on the basis of the spirit of the invention. Additionally, the modifications are included in the scope of the invention.

What is claimed is:

1. A neutron capture therapy apparatus comprising:
an irradiator that irradiates an irradiation spot of an irradiation target with a neutron ray; and
an electromagnetic wave irradiator that irradiates the irradiation target with an electromagnetic wave for aligning the irradiation target with a position where the irradiation target is disposed, wherein
the electromagnetic wave irradiator changes an irradiation position of the electromagnetic wave based on treatment plan information regarding the irradiation target.

2. The neutron capture therapy apparatus according to claim 1, further comprising:
an accelerator that accelerates charged particles of negative ions and emits a charged particle beam.

3. The neutron capture therapy apparatus according to claim 2, further comprising:
a neutron ray generating unit to which the charged particle beam emitted from the accelerator is sent.

4. The neutron capture therapy apparatus according to claim 3, wherein
the neutron ray generating unit includes a beam duct and a target, and the charged particle beam emitted from the accelerator passes through the beam duct and proceeds toward the target.

5. The neutron capture therapy apparatus according to claim 1, further comprising:
a control device that controls an entirety of the neutron capture therapy apparatus.

6. The neutron capture therapy apparatus according to claim 5, wherein
the control device includes a treatment plan acquisition unit, a laser position calculation unit, and an operation control unit, and
the treatment plan acquisition unit acquires treatment plan information including information on a distance and a direction between a reference position on a side of the irradiator and the irradiation spot.

7. The neutron capture therapy apparatus according to claim 6, wherein
the laser position calculation unit calculates an irradiation position of a laser based on the treatment plan information acquired by the treatment plan acquisition unit.

8. The neutron capture therapy apparatus according to claim 7, wherein
the operation control unit controls a laser irradiator, which is a laser irradiation unit, to move based on a calculation result by the laser position calculation unit.

9. The neutron capture therapy apparatus according to claim 1, wherein
the electromagnetic wave irradiator is a laser irradiation unit.

10. The neutron capture therapy apparatus according to claim 1, wherein
the treatment plan information includes at least information for calculating a distance and a direction between a reference position on a side of the irradiator that emits the neutron ray and the irradiation spot.

11. The neutron capture therapy apparatus according to claim 1, wherein
the electromagnetic wave irradiator changes the irradiation position of the electromagnetic wave along an axial direction in which a center axis of the neutron ray extends.

12. The neutron capture therapy apparatus according to claim 1, wherein
the electromagnetic wave irradiator changes the irradiation position along a direction perpendicular to an axial direction in which a center axis of the neutron ray extends.

13. The neutron capture therapy apparatus according to claim 1, wherein
the irradiation position of the electromagnetic wave irradiator is recalculated based on information regarding an irradiation field forming device that forms an irradiation field of the neutron ray.
